(19) United States
(12) Patent Application Publication
(10) Pub. No.: US 2014/0196726 A1
(43) Pub. Date: Jul. 17, 2014

(54) INTEGRAL MOUTHGUARD OXYGEN MASK

(71) Applicant: Mark L. Mallek, Nashua, NH (US)

(72) Inventor: Mark L. Mallek, Nashua, NH (US)

(21) Appl. No.: 13/741,933

(22) Filed: Jan. 15, 2013

(51) Int. Cl. A61M 16/06; A61M 16/00; A61M 16/08; A61C 5/14

(52) U.S. Cl. CPC A61M 16/06; A61C 5/14; A61M 16/0057; A61M 16/0875
USPC 128/861; 29/428

(57) ABSTRACT

A single use, disposable, oxygen mask having an integral mouthguard/bite-block, and an oxygen intake port having a right angle is placed centrally on a side of the mask along with a right angle port to facilitate an endoscopic procedure. A patient biting down on the bite-block portion of the mouthguard keeps the mask in place. The mask has a concave shaped body that fits over and around the nose and mouth of the patient. An exhaler side port is provided on an opposite side of the mask.

### BACKGROUND OF THE INVENTION

1. Field of the Invention

This invention relates generally to oxygen masks and, in particular, to a one piece, disposable, oxygen mask having an integral mouthguard/bite-block and an oxygen intake having a right angle port to allow lateral access of oxygen tubing on the side of the mask to facilitate short endoscopic procedures.

2. Description of Related Art

U.S. Pat. No. 4,470,413 issued Sep. 11, 1984, to Warncke discloses a protective breathing apparatus including a mask, a mouthpiece, the mask having a thickened area on each side of a person's nose to constrict the nose so that breathing is through the mouth. However, this prior art patent does not teach or disclose the structure of the present invention, and particularly, it is nonendoscopic.

U.S. Pat. No. 5,431,158 issued Jul. 11, 1995 to Tirotta discloses an endoscopy breathing mask having a bite-block attached to the mask with a central opening adapted to receive a scope therein for use in endoscopy. It requires a head strap for positioning on a patient, has a beaded edge surrounding the mask to provide rigidity, and it has the cleared areas on each side of the mask to constrict the nose of the wearer so breathing is through the mouth. However, it does not teach or disclose the structure of the present invention such as having an opening immediately under a wearer's nose and no nose constriction.

U.S. Patent Application Publication No. US 2008/0053449 published Mar. 6, 2008 for Scott Lindblom et al discloses a respiratory mask for use in endoscopic or bronchoscopic procedures having a shell defining a cavity therein and an inflatable patient cushion extending around the perimeter of the shell, an inlet tube for oxygen, two spaced apart ports on the lower portion of the shell each including a perforated diaphragm, and a third port in the upper portion of the shell for receiving a monitoring probe. However, it does not teach or disclose the bite-block mouthguard and the nose area structure of the present invention.

### SUMMARY OF THE INVENTION

Accordingly, it is therefore an object of this invention to provide a single use, disposable, oxygen mask having an integral mouthguard/bite block and an oxygen intake with a right angle port to facilitate attachment of an oxygen supply line to enable endoscopic procedures while supplemental oxygen is simultaneously delivered.

It is another object of this invention to eliminate the need for an elastic head strap by having the integral mouthguard/bite-block and to eliminate any metal nasal bridge adjuster by having a flatter, narrower, nose space within the mask.

It is a further object of this invention to provide all necessary openings in the mask normally needed by an anesthesiologist especially when endotracheal intubation is not required.

These and other objects are further accomplished by an oxygen mask to be worn by a patient with a concave shaped body adapted to fit over the mouth and nose of the patient, a mouthguard having a bite-block extending within the mask, the mouthguard being located in a lower portion of the concave shaped body, and a first port positioned approximately midway along the length of the shaped body adjacent to a nose area for receiving oxygen. The first port comprises a right angle portion for attaching a tube from a source of oxygen. The bite-block has a flange on an end extending within the mask. The mask has a second port positioned approximately midway along the length of the shaped body adjacent to the nose on an opposite side from the first port.

These objects are further accomplished by a method of making a disposable oxygen mask to be worn by a patient comprising the steps of forming a concave shaped body to fit over the mouth and nose of the patient, providing a mouthguard within the mask, the mouthguard having a bite-block extending a predetermined distance within the mask, the mouthguard being located in a lower portion of the concave shaped body, and positioning a first port approximately midway along the length of the shaped body adjacent to a nose area for receiving oxygen. The step of positioning a first port includes providing the first port with a right angle portion for attaching a tube from a source of oxygen. The step of providing a mouthguard having a bite-block comprises the step of providing a flange on an end of the bite-block extending within the mask. The method further comprises the step of providing a second port positioned approximately midway along the length of the shaped body adjacent to the nose area on an opposite side from the first port.

Additional objects, features and advantages of the invention will become apparent to those skilled in the art upon consideration of the following detailed description of the preferred embodiment exemplifying the best mode of carrying out the invention as presently perceived.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a front elevational view of an integral mouthguard oxygen mask according to the present invention.

FIG. 2 is a left side perspective view of the oxygen mask showing the integral mouthguard/bite-block and oxygen intake port.

FIG. 3 is a right side perspective view of the integral mouthguard oxygen mask showing a side port and the integral mouthguard/bite-block.

### DESCRIPTION OF ILLUSTRATIVE EMBODIMENT

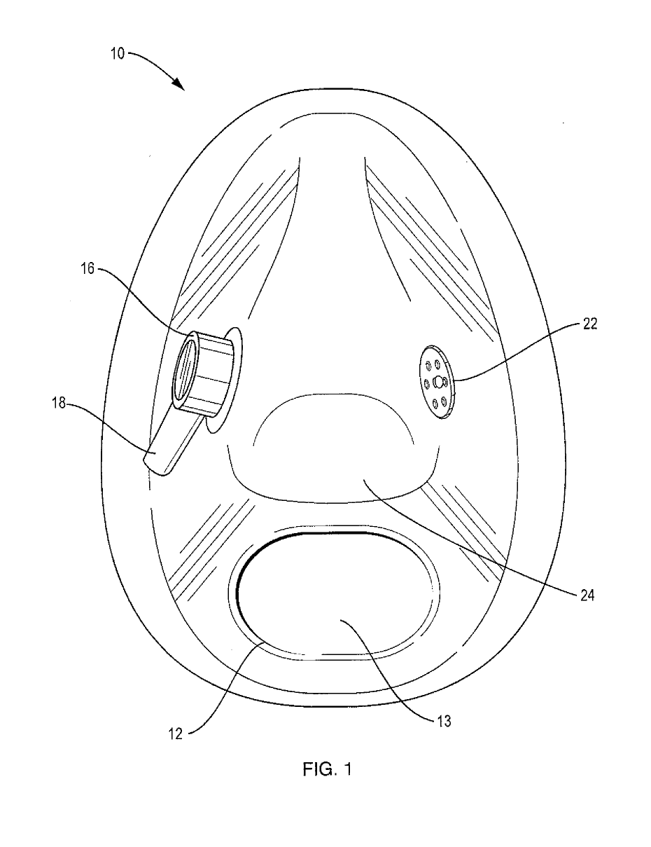

Referring to FIG. 1, a front elevational view of a disposable, integral mouthguard oxygen mask 10 is shown according to the present invention. In particular, a mouthguard 12 having a bite-block 11 portion is formed into a center, lower portion of the mask 10 so that the opening 13 of the mouthguard 12 will be located opposite a patient's or wearer's mouth. A patient biting down on the bite-block 11 keeps the mask 10 in place. An oxygen source either from a wall supply, or oxygen tank connects to the mask 10 via tubing to a right angle portion 18 of a port 16 located centrally on a left side area of the oxygen mask 10 spaced away from the mouthguard opening 13.

The oxygen mask 10 is used during endoscopic and other types of medical procedures during which a patient is receiving sedation, but does not require full anesthesia nor edotracheal intubation, yet oxygen is used. The mask 10 allows access to the patient's patient's mouth so that an instrument such as an endoscope may be passed and oxygen delivered at the same time.

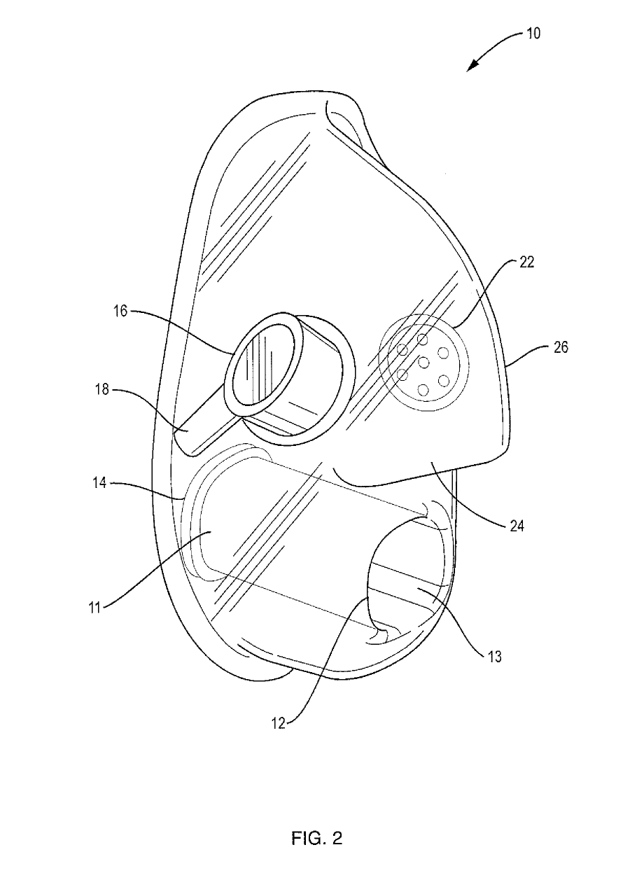

Referring to FIG. 1 and FIG. 2, FIG. 2 is a left side perspective view of the oxygen mask 10 showing the integral mouthguard 12 having an opening 13 and a flange 14 located on an inner end of the bite-block 11. The flange 14 provides support for retaining a patient's teeth on the oval shaped bite-block 11 when positioned within patient's mouth. The flange 14 end of the bite-block 11 in the preferred embodiment has a height of approximately 1¼ inches and a width of approximately 1 inch. The mouthguard/bite-block 12 which is oval shaped has a height of approximately 1 inch, a width of approximately ¾ inches and a depth of approximately 2 inches. The mask 10 is one piece and it is made of clear, polyvinyl chloride (PVC). The mouthguard/bite-block 12 is molded as an integral part of the one piece mask 10. The oxygen intake port 16 is attached to the side of the mask 10 over a circular area having approximately seven holes to enable oxygen to pass into the mask 10 when on a patient. The oxygen intake port 16 is approximately one-half inch in diameter and ¾ inch long. A right angle portion 18 is provided on the side of the oxygen intake port 16 to facilitate attachment of an oxygen supply line which is typically tubular extending from a wall mounted oxygen supply or oxygen tank. This right angle portion 18 of port 16 is particularly useful when a patient is lying on his left side when having a gastroscopy; otherwise, the oxygen tubing and port 16 might get depressed into the patient's pillows. The oxygen mask 10 is particularly useful to facilitate an endoscope entry into the mouth and to allow oxygen during a procedure and not as a respiratory aid. The slope of the mask nose area 26 has a shallow convexity conforming to the shape of a typical human nose and allowing sufficient space to receive the nose comfortably.

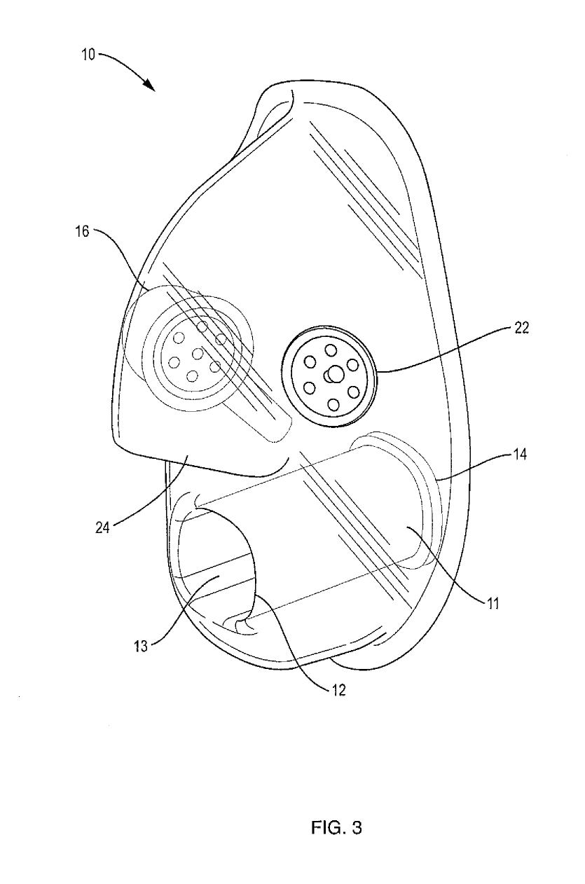

Referring to FIG. 3, a right side perspective view of the integral mouthguard oxygen mask 10 shows a side exhale port 22 having a circular shape with a plurality of holes (seven) to allow carbon dioxide rich exhaled air to leave the mask and by means of a flap valve (not shown) to block room/ambient air from returning into the mask 10. The side port 22 is located approximately midway between the nose area 26 and the ridged edge of the oxygen mask 10.

This invention has been disclosed in terms of a particular embodiment. It will be apparent that many modifications can be made to the disclosed apparatus without departing from the invention. Therefore, it is the intent of the appended claims to cover all such variations and modifications as come within the true spirit and scope of this invention.

What is claimed is:

1. An oxygen mask to be worn by a patient comprising:
a concave shaped body adapted to fit over the mouth and nose of said patient;
a mouthguard having a bite-block extending within said mask, said mouthguard being located in a lower portion of said concave shaped body; and
a first port positioned approximately midway along the length of said shaped body adjacent to nose area for receiving oxygen.

2. The oxygen mask as recited in claim 1 wherein said first port comprises a right angle portion for attaching a tube from a source of oxygen.

3. The oxygen mask as recited in claim 1 wherein said bite-block comprises a flange on an end extending within said mask.

4. The oxygen mask as recited in claim 1 wherein said mask comprises a second port positioned approximately midway along the length of said shaped body adjacent to said nose area on an opposite side from said first port.

5. A method of making a disposable oxygen mask to be worn by a patient comprising the steps of:
forming a concave shaped body to fit over the mouth and nose of said patient;
providing a mouthguard within said mask, said mouthguard having a bite-block extending a predetermined distance within said mask, said mouthguard being located in a lower portion of said concave shaped body; and
positioning a first port approximately midway along the length of said shaped body adjacent to a nose area for receiving oxygen.

6. The method as recited in claim 5 wherein said step of positioning a first port includes providing said first port with a right angle portion for attaching a tube from a source of oxygen.

7. The method as recited in claim 5 wherein said step of providing a mouthguard having a bite-block comprises the step of providing a flange on an end of said bite-block extending within said mask.

8. The method as recited in claim 5 wherein said method comprises the step of providing a second port positioned approximately midway along the length of said shaped body adjacent to said nose area on an opposite side from said first port.
